(19) United States
(12) Patent Application Publication
(10) Pub. No.: US 2026/0346224 A1
(43) Pub. Date: Nov. 5, 2026

(54) REACTORS IN A MICROSTRUCTURE ARRANGEMENT

(71) Applicant: INERATEC GMBH, Karlsruhe (DE)

(72) Inventors: Florian BETZNER, Karlsruhe (DE); Peter PFEIFER, Linkenheim-Hochstetten (DE); Sebastian FICHTNER, Heidelberg (DE)

(21) Appl. No.: 19/115,542

(22) PCT Filed: Sep. 27, 2023
(86) PCT No.: PCT/EP2023/076716

§ 371 Date: Mar. 26, 2025

(30) Foreign Application Priority Data
Sep. 30, 2022 (DE) 102022125342.2

(51) Int. Cl. B01J 19/24; C07C 1/02

(52) U.S. Cl. CPC B01J 19/249; C07C 1/02; B01J 2219/00085; B01J 2219/2453; B01J 2219/2458; B01J 2219/2462; B01J 2219/2485; B01J 2219/2493

(57) ABSTRACT

The present invention relates to a reactor with a multilayer structure with a desired number of, arranged one on top of the other and/or next to one another and connected to one another, individual segments made of diffusion-welded stacks of microstructured metal plates, in which the distribution channels for the coolant are deflected at least twice, and wherein the coolant is initially introduced laterally in cross-flow exclusively into end metal plates of the stacks, transversely to the flow direction of the process fluid, and thereafter the coolant is deflected in the stack direction and distributed to several reaction areas within a segment; and a method for producing reactors, preferably (micro-)structured reactors, comprising layers for, on the one hand, the flow of reaction medium and, on the other hand, the flow of heat transfer medium

All documents cited in the present application are incorporated by reference in their entirety in the present disclosure.

The present invention relates to a reactor with a multilayer structure with a desired number of individual segments arranged on top of and/or next to one another and connected to one another, made of diffusion-welded stacks of microstructured metal plates, in which the distribution channels for the coolant are preferably deflected at least twice, and in which the coolant is preferably first introduced laterally in cross-flow exclusively into regions resulting from end metal plates of the stacks, transversely to the flow direction of the process fluid, and then the coolant is deflected in the stack direction and distributed over several reaction regions within a segment, and a method for producing reactors, preferably (micro-)structured reactors, comprising layers for, on the one hand, the flow of reaction medium and, on the other hand, the flow of heat transfer medium

### State of the Art

In (micro-)structured reactors, solid catalysts can be introduced as layers or as fillings of particles. Such reactors are usually used for fast and high-energy reactions in order to ensure an efficient heat supply or removal with the help of a layer structure, i.e. by alternating layers with flow of reaction medium and heat transfer medium, and thus to operate the reaction at a temperature setpoint if possible.

When applying such microstructured reactors, it is known that it is important that all structures, whether slot-shaped or channel-shaped, are uniformly flowed through. In addition to the influence of manufacturing tolerances on the effective hydraulic diameter, which significantly affects the flow resistance and thus, conversely, the volume flow in the respective parallel structures when the inlet and outlet are common (Pfeifer et al., Characterization of flow distribution in microchannel reactors, AIChEJ, 418-425, 2004), the flow conditions on the plurality of structures are also relevant for the uniform application of the working fluid (Pfeifer et al., Hot wire anemometry for experimental determination of flow distribution in multilayer microreactors, Chem Eng J 135S (2008 ) pages 173-178). If the parallel structures are filled with catalytically active material, a particle size distribution that is as narrow as possible is advantageous, so that the local porosity in the entire particle heap is as identical as possible when filling and thus a uniform distribution of the mass flow over the parallel structures is possible. With a correspondingly high pressure drop in the particle heap, external flow conditions become almost irrelevant for the uniform distribution.

This is also described in U.S. Pat. No. 10,335,759 B1, in which ultrasound and fluidisation of the particle bed are used to achieve the most homogeneous distribution of the catalyst particles possible. In addition, however, the size of the channels in which the particles are introduced is just as important for the even distribution of the material flow, as well as the channels running in parallel thereto for cooling or heating the reaction. In the particle-filled channel, the ratio of volume in which particles are in contact with the wall to volume in which particle-particle-contacts are present increases as the channel becomes smaller. This applies in particular to a channel-to-particle size ratio of 2-10, which can occur in microchannels. This leads to the fact that even under conditions in which the particle structure becomes a major influencing factor, the size tolerance of the channels themselves has an influence on the uniform distribution. This applies in particular to very fast reactions in which a very short residence time is required and the catalyst bed length is rather small. If some adjacent channels for cooling or heating the reaction are smaller than others, this leads to the fact that there is less mass flow for thermostatizing the reaction at these locations, which can have a negative effect on the desired temperature or temperature profile in the reactor.

When manufacturing microstructured reactors, the parallelisation of channels optimized for heat and mass transfer leads to an increase in throughput. Ideally, the throughput increases with the number of channels, provided that the mass flow is evenly distributed. It is clear that when the channels are parallelised, the heat transfer to adjacent channels must always be considered, because these must also be supplied with medium. Cross-flow design is usually best suited for this purpose, since in this design the connection at the side surface of the reactor is possible. Corresponding designs are known from many patents, see also DE 10 2005 022 985 and U.S. Pat. No. 5,249,359. The supply of coolant in countercurrent or cocurrent flow arrangement geometrically forces a relatively large area of the channels to operate in cross-flow, where heat transfer is already taking place. This increases the coolant temperature from the entry of the heat transfer medium to the location where the area of the counterflow or co-current flow is to be. In this respect, the supply of coolant of the same temperature for counterflow or co-current flow depends on the number of reaction channels that are crossed in the design. This is problematic in particular for very fast reactions with high heat of reaction. This problem is further complicated by a deliberately desired reduction of pressure loss in microchannels with a packed bed by shortening the bed length and thus consequently also shortening the heat exchange zone. In this respect, the enlargement of the external dimensions of a plate package with microchannels for reaction and integrated cooling is severely limited in width and length.

Since the plate pack also has a height limitation, the parallelisation of reactor modules is often proposed. The limitation arises from the connection technology of a module, either due to increasing stresses when welding multilayer stacks of metallic plates using a blasting process or due to a consumption and possibly shearing of a metallic plate stack due to the application of force to the stack during diffusion welding.

The necessity of using a plurality of reactor modules to achieve high plant capacities has disadvantages with regard to the necessary piping and the flow to individual reactor modules. Due to cost advantages in pumps or compressors with the scaling of the throughput, a common supply of the individual modules is advantageous. US 2014/0357738 A1 therefore proposes an integration of the individual metallic reactor modules into a common pressure vessel, in which internal sealing of the individual connection areas of the channels with respect to the distribution chambers is carried out.

When manufacturing the individual reactor modules and the microchannels produced on the plates or via cutouts, tolerances in size are to be expected, in particular when using a plurality of individual plates, due to the rolling of the plates, as well as due to the joining of the plates by individual diffusion welding processes. As described above, this leads to a possible uneven distribution of the process fluid or the coolant.

EP 3 463 642 B1 (in which, if several reactors are used, they are connected in series), DE 103 39 972 A1 (in which connections are obtained by laser welding) could be mentioned as further state of the art; or, possibly, (technically more remotely) DE 60 2004 009 360T2 , DE 103 18 257 A1, EP 1 352 686 A1, DE 10 2004 037 059 B4.

In this respect, there is still a considerable need to improve the previous state of the art, based on the known state of the art.

### OBJECT

The object of the present invention was therefore to overcome the disadvantages of the state of the art described above and to provide reactors that no longer have these problems, or at least only to a considerably lesser extent.

A reactor design that avoids the disadvantages of the prior art and improves the known reactors should be found.

In addition, a manufacturing process should be found that allows such reactors to be produced comparatively easily, safely and economically.

Further objects arise for the person skilled in the art when considering the claims and from the following description.

### Solution

These and other objects that become apparent for the person skilled in the art from the present description are solved by the subject matter presented in the claims, with the dependent claims representing preferred and particularly advantageous embodiments. In the context of the present invention, the front side of the reactor is understood to be the side from which the reactor is supplied with reaction medium/fluid, and the back side of the reactor is understood to be the side from which the reaction medium/fluid exits the reactor.

### DESCRIPTION

Subject matter of the present invention is, in particular, a method for producing reactors, preferably (micro-)structured reactors, comprising layers for, on the one hand, the flow of reaction medium and, on the other hand, the flow of heat transfer medium.

For that, in step a), structured metal plates, each having the same width and the same depth, are first stacked on top of one another for the respective layers. Preferably, the metal plates each have edge lengths of 20 cm to 200 cm. In preferred embodiments, the metal plates have thicknesses of 0.3 mm to 12 mm.

In optional embodiments, which are preferred in some variants, (a) structured or non-structured metal plate(s) is/are also stacked as the uppermost and/or lowermost end metal plate(s) (sometimes referred to as end metal plate or end plate).

The orientation of the metal plates, in particular the classification of what is the back and front or what the sides of the individual metal plates are, results from the structure of the metal plates, which form the reactor structure itself, by incorporated reaction channels or groups of reaction channels. This is because the reaction medium flows through the reactor structure (reaction channels) from one side-the front side-to the other side-the back side. The orientations of the other metal plates inevitably result from the metal plates containing the reactor structures. This is readily known to those skilled in the art.

In the next process step b), the plates stacked in this way are joined together by diffusion welding.

In preferred embodiments of the present invention, the height of the plate stacks in this context is at most 1.5 times greater than the longest edge length (for example, with an edge length of 20 cm, the stack would be at most 30 cm high).

Next, in step c), the diffusion-welded stack obtained in this way is divided into individual segments of equal depth in the width direction. In some preferred embodiments of the present invention, the individual segments have a depth between 3 cm and 30 cm, in others a depth between 3.5 cm and 32 cm. It should be noted here that the reaction medium can also be diverted between these two sides and does not necessarily flow in a straight line between the two sides. In addition to strictly straight reaction channels, reaction channels in other configurations are also conceivable, preferably jagged, wavy or meandering, with loops. However, straight reaction channels are most preferred, especially for reactions with a short residence time.

In some embodiments, the depth of the segments equals the distance between the front and back of the reactor.
This applies in particular if structured metal plates are used in which the reaction channels extend to the edge of the plate.
This also applies when structured metal plates are used in which the reaction channels do not extend to the edge of the plate and the front or back sides of the segments have to be milled down after production until the reaction channels are reached, but milling is not carried out in the edge area and a circumferential edge is left standing (this promotes stability and is excellently suited to carry flange connections for the common supply of reaction medium). This procedure is preferred according to the invention because, among other things, it offers a good compromise between ease of manufacture, stability and functionality. This milling can optionally be carried out before or after step d) described below. In some embodiments, it is useful and therefore preferred to mill after step d), since any unevenness from the connection of the segments can then be levelled out.
However, this does not apply if structured metal plates are used in which the reaction channels do not extend to the edge of the plate and the front and back sides of the segments have to be milled down after production until the reaction channels are reached, and the surfaces are completely milled and no surrounding edge is left.

Then, in the next process step d), a desired number of individual segments obtained in the previous step are arranged on top of and/or next to each other and connected to each other. Preferably, a lower end plate of a segment is arranged on an upper end plate of a further segment if the sequence of the plates in the stack is symmetrical, i.e. the sequence seen from bottom to top does not differ from a view from top to bottom. In other variants, an upper end plate can be arranged on an upper end plate of a further segment, or a lower end plate can be arranged on a lower end plate of a further segment; this makes a difference if the sequence of the plates in the stack is different. In some preferred embodiments, only an arrangement on top of each other is made. The connections can be made by any method, preferably selected from screwing, clamping, gluing or circumferential welding, in particular circumferential welding.

On top of each other therefore means that the segments are arranged end plate-to-end plate and next to each other means side-by-side.

It should be noted that in the case of individual segments being manufactured by means of diffusion welding, a maximum height of three times the segment depth is difficult to achieve (i.e. the resulting reactors would no longer be usable) or would lead to a high degree of bulging on a segment. If the height of the plate stack is greater than their width as the largest side length by a factor of 1.5 and, in step d), for example, 5 segments are arranged on top of each other, a factor of 7.5 for the maximum height of the assembled reactor relative to its width results.

In preferred embodiments of the present invention, before or simultaneously with step c), preferably before step a), recesses are incorporated into the metal plates or the stack along which the division is then carried out in step c). Preferably, these can be produced by etching, milling, blanking or other means; in some (further) preferred variants by blanking.

The position of this row of recesses thus defines the segment depth in the finished product.

In some preferred variants of the present invention, blankings are incorporated at the same height as the recesses on the sides of the metal plates or on the sides of the stack. This makes it the separation into segments even easier.

To make the separation easier, the recesses are preferably as long and narrow as possible but should not exceed a certain length. The maximum length is based on the desired width of individual reactor sections (i.e. respective groups of reaction channels, which can also be referred to as (reactor) modules, even if they only refer to a respective structured metal plate), i.e. the width of the respective reactor openings (respective groups of reaction channels), as specified by the metal plates. In this context, it is preferred if the reaction zones resulting from the reactor sections in the finished reactor can still be easily cooled in parallel or co-current flow when considered individually. The length of individual recesses is smaller than the depth of the segment depth by a factor of 2 in some variants. In particularly preferred embodiments, the lengths of the recesses are in the range of 1.5 to 15 cm. In preferred embodiments of the present invention, thus one or, more preferably, two or several of these recesses are incorporated into the metal plates or the stack, wherein the ratio of the sum of the recess lengths to the total width of the metal plates preferably is from 60% to 90%, more preferably from 30% to 70%. In some preferred variants, two or at least two recesses are incorporated, since in these variants it may also be a matter of achieving a parallelisation of individual reactor modules incorporated in the plates.

In preferred embodiments of the present invention, a ratio of segment depth to recess length (=width of the reactor module) of 2 to 8 is adhered to. It is also preferred that the reaction zone length (length of the reaction channels) is greater than a single recess length, i.e. the respective width of a reactor section. In some preferred embodiments, at least one recess zone in the respective metal plate is located “above” each reaction. The recesses are preferably long enough for all channels of a reactor module to be located within this recess, i.e. the length of the recess is greater than the width of the area in which the channel outlets are located (i.e. greater than one module width). The total width, as the sum of all recesses and the intermediate areas, is nevertheless significantly greater than the reaction zone length due to the number of reaction zones in a segment (section), in particular if the segments (sections) have more than two recesses or reactor modules.

In preferred embodiments of the present invention, the metal plates used in step a) are approximately square, preferably exactly square. Approximately square means an aspect ratio of 1:2 to a maximum of 2:1, preferably of 1:1.3 to 1.3:1, more preferably 1.15:1 to 1:1.15, particularly preferred 1.1:1 to 1:1.1 and in particular preferred exactly an aspect ratio of 1:1. Of course, manufacturing tolerances may lead to a slightly different aspect ratio.

In further preferred embodiments of the present invention, the connections of the individual segments obtained in step d) are planarised to create planar sealing edges, in the case of circumferential welding in step d) preferably by milling the resulting weld seams.

In further preferred embodiments of the present invention, the structured metal plates have an edge all around their structuring (among other things, so that fewer deformations occur during diffusion welding) and after step c) or after step d), preferably after step d), a step f) (‘f’ as in “fräsen”, the German word for milling), in which the front and back sides of the segments are milled down to the openings of the reaction channels. The milling can be done over the entire surface or in such a way that a continuous edge remains in the resulting reactor. The circumferential edge is preferably somewhat thinner than the side edge of the structured metal plates (there is still a (small) clearance between the lateral end of the respective structuring and the inner side of the circumferential edge).

In still further preferred embodiments of the present invention, the flat sealing edges are each adapted with a flange or all together with a flange or partly.

In preferred embodiments of the present invention, the end metal plates are selected and designed so that the coolant can be introduced into their interior in a cross-flow perpendicular to the flow direction of the process fluid. The coolant channels in the end metal plates are further configured so that the coolant is diverted parallel to the stacking height of the segments.

It is further preferred that there be at least one at least two-fold deflection of the coolant within the distribution channels in the reactor. In particularly preferred exemplary embodiments, the first cross-flow area is achieved with channels in the dimension 5 mm to 20 mm. In these embodiments, the sub-distribution in the stacking direction, spatially viewed besides the individual reactor modules, is achieved via channels in the dimension 2 mm to 10 mm and after the second deflection back to cross-flow then with channels of 2 mm to 5 mm. The actual channels for thermostatizing, which run parallel to the reaction channels, typically have a diameter and/or edge length of <2 mm in these designs.

In preferred embodiments of the present invention, the end metal plates are selected such that the coolant can flow in cross-current through areas without proximity to reaction channels. The term ‘proximity’ here preferably means a distance of at least 5 mm, preferably between 5 mm and 10 mm.

In preferred embodiments of the present invention, the end metal plates, if present, have a thickness of equal to or greater than 5 mm, preferably 10 mm, and/or less than 80 mm, preferably 70 mm; more preferably between 10 mm and 70 mm, in some other embodiments between 10 and 40 mm, and inlet openings for coolant. In some preferred embodiments, these inlet openings can have a diameter of 5 to 20 mm (of course, the diameter of the inlet openings is to be adapted depending on the plate thickness in such a way that the plates and openings or channels are stable and closed; this is readily apparent to and adaptable by a person skilled in the art).

Further preferred embodiments are that the end metal plates have drillings in the same locations.

In some preferred embodiments, heating elements are also inserted in the stacking direction. For this purpose, preferred embodiments already provide round recesses in the end plates and the plate elements, which can be further drilled after diffusion welding and after the connection of the segments, if necessary, to achieve a high fitting accuracy between heating element and metal plates in the case of deforming cutouts due to diffusion welding and due to possible offset when connecting the segments. Alternatively, but usually less preferably, it is also possible within the context of the present invention to incorporate continuous drillings only after the connection of the segments. The round recesses are arranged in each case so that they are located beside or between the reactor zones, i.e. so that no connection is created to the branches of the cooling channels.

Another subject matter of the present invention is, in particular, a reactor which is characterized in in that it has a desired number of individual segments which are arranged one on top of the other and/or next to one another and are connected to one another, in particular one on top of the other, wherein a lower end plate of a segment is arranged on an upper end plate of a further segment, made of diffusion-welded stacks of microstructured metal plates, in which the distribution channels for the coolant are deflected at least twice, and wherein
the coolant is initially introduced laterally in cross-flow exclusively into end metal plates of the stacks, transversely to the flow direction of the process fluid,
thereafter in the end metal plates the coolant is deflected in the stack direction and distributed to several reaction areas within a segment.

Preferably, the reactor according to the invention is produced according to the method described above.

Not least, the use of a reactor according to the invention or a reactor produced by the method according to the invention for Fischer-Tropsch reactions or methanisation reactions is in particular the subject of the present invention.

With regard to the present invention and its advantages, the following can be stated:

Compared to several smaller individual modules, an increase in the plate dimensions and thus of the entire welded body is advantageous in order to reduce the influence of the tolerances due to the rolling process of the plate base material, as well as compared to several individual diffusion welding processes of individual modules. However, this is not easily possible due to size limitations. Since the reactor width usually has to be significantly larger than the reactor length for very fast reactions, individual segments are rectangular in shape. According to the invention, several such shapes are arranged in a row until an approximately square shape for diffusion welding is achieved and then separated into individual sections after the diffusion welding process. Due to the square shape the plate stack can be higher than with a rectangular shape (risk of shearing is reduced), which is advantageous for the number of reactor modules. By rotating the individual cut segments by 90° to each other, an even greater stack height can be achieved by lining them up and performing a corresponding circumferential welding on the contact surfaces, whereby the process fluid can enter and exit the exposed channels at the resulting end faces. The invention also includes the provision of blankings at the locations where the welded body is separated in order to facilitate the separation. The recesses should be as narrow and long as possible; ideally, the ratio of recess lengths to the total width of the plates should be 30-70%. Furthermore, it is preferable to have at least two recesses rather than a single one. A ratio of 2 to 8 between the length of the reaction zone and the length of the recess has proven to be particularly effective in preventing the stack of plates from being bulged in the recesses. Since all segments are made from a single plate stack, the tolerances from the rolling process, as well as those resulting from the diffusion welding process, are reduced. Even if, in the case of using the same welding parameters, different deformations of the individual plate stacks are obtained (inaccurate temperature measurement at high temperatures, deviations in the steel structure, etc.), the entire structure that is created can be adapted by circumferential welding at the end faces, after the welds have been milled over to produce flat sealing edges, with a flange in each case, so that the effort required for connections is significantly reduced compared to many individual modules.

In order to achieve the widest possible reactor width, the invention allows for the coolant to be deflected at least twice within distribution channels as wide as possible, whereby the coolant is initially introduced laterally (cross-flow) and exclusively into the end metal plates perpendicular to the flow direction of the process fluid. It is then deflected in the stacking direction and distributed to several reaction areas within a separated reactor segment. The channelling in the end metal plates, which in preferred embodiments have a thickness of 10 mm to 40 mm, as well as the channelling in the plate stack direction in areas without reaction channels, prevents significant heat exchange until the coolant enters on the individual plates in the cooling channels. Finally, a sufficiently high ratio of the length of the catalyst bed to the width of the reaction area ensures that the countercurrent/co-current flow area is maximised. If the end metal plates of the reactor segments are placed beside each other as described above, the introduction of heat into the supply lines of the coolant is minimal.

With the present invention, a maximization of the reactor size can be achieved with very short reaction channels in the range of 2.5 cm to 28 cm or also 3 cm to 30 cm in length (these sizes correlate with the above-mentioned depths of the individual segments, depending on whether (over)milling is (has to be) carried out or not—if milling is necessary, the segment depths are slightly greater than the reactor channel lengths).

The present invention is therefore based in particular on an optimized manufacturing process for parallelized multiple modules, which could not be derived from the prior art, whereby it was particularly surprising that the inventive approach (and resulting products) led to excellent results.

With the present invention, the limitations of reactor size in terms of reactor width and stack height for reactions with very short residence times, i.e., very short reaction zones, are greatly reduced. At the same time, the distribution of coolant flows is optimised, and the uniform distribution of the process fluid is optimised.

In the context of the present invention, in particular the preferred embodiments, a parallelization of several modules for very short residence times is achieved without the need for control fittings or residence elements. The present invention (i.e. in particular the parallelization of modules, which initially arise from the fact that the modules are decollated from a plate stack and in which one of the material flows is distributed over the modules by rotating them through 90° and following connecting technique) avoids or eliminates the fundamental difficulties in constructing altogether large equipment with a very short side length (see above). The manufacturing ‘trick’ according to the present invention results in significant economic advantages for plant engineering in the saving of such fittings.

The skilled person can carry out the exact design of the devices described, insofar as these are not explicitly described in this description, such as size, wall thicknesses, materials, etc., for the reaction conditions envisaged for a particular reaction, within the scope of his general expertise.

If, in the description of the devices according to the invention, parts or the entire device are marked as ‘consisting of’, this is to be understood as referring to the essential parts mentioned. Obvious or inherent parts such as pipes, valves, screws, housings, measuring devices, storage containers for educts/products, etc. are not excluded.

The individual parts of the devices are, unless explicitly described, operatively connected to each other in the usual and known manner.

The various embodiments of the present invention, e.g.—but not limited to—those of the various dependent claims, can be combined with each other in any way, provided that such combinations do not contradict each other.

The present invention will be explained in more detail below with reference to the drawings. The drawings are not to be interpreted in a limiting way and are not to scale. The drawings are schematic and furthermore do not contain all the features that are present in conventional devices but are reduced to the features that are essential for the present invention and for understanding it; for example, screws, connections, etc. are not shown or are not shown in detail.

The same reference signs show the same features in the figures, the description and the claims.

In the context of the following explanations, some variants or advantageous and preferred variants/embodiments are also presented which, unless otherwise stated, are not limited to the respectively described figure, but are preferred variants of the present invention per se (unless contradictory).

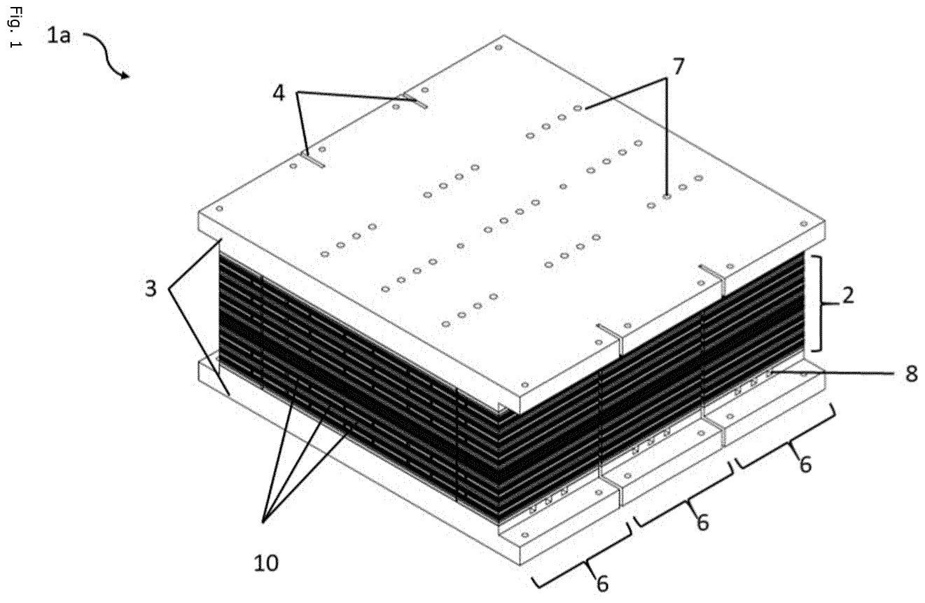

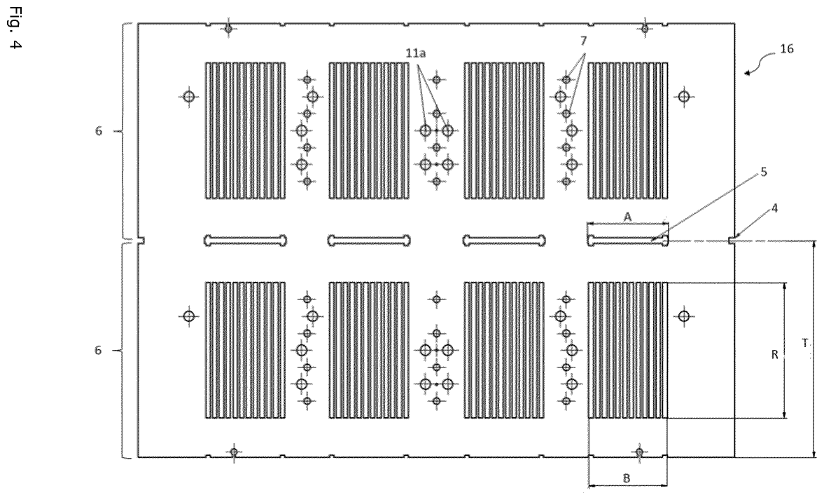

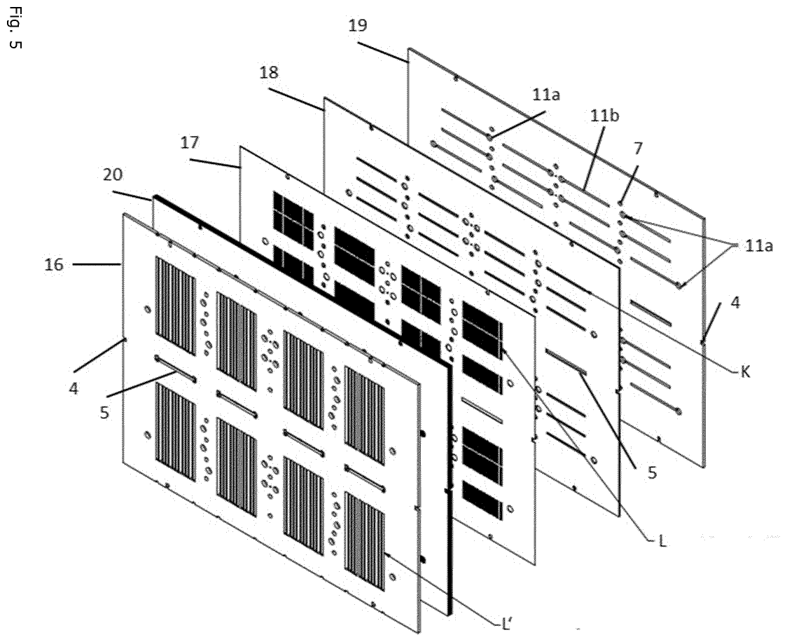

FIG. 1 shows a reactor 1a according to the invention, in the form of a stack 2 of structured metal plates arranged on top of each other with metal end metal plates 3 at the top and bottom. The plates are all diffusion-welded as a whole. The reactor is shown before being separated into the individual segments 6 (there are three in the example shown). Also visible are the blankings 4 along which the segments 6 are separated from one another; the recesses 5 which are also intended for separation are not visible here, since these are not present in the cover plates, but only in the stacking plates (metal plates) in between (see also FIGS. 4 and 5). Also indicated are reactor openings 10 in the individual structured metal plates. If desired, catalyst can be arranged in these during operation of the reactor. Furthermore, holes 7 for heating elements are shown in the metal end metal plates. The inlet openings 8 for coolant, which are located here laterally in the end metal plates 3, can also be seen.

It should be noted that FIG. 1 shows an embodiment in which structured metal plates are used for the stack 2, which have reactor openings 10 on the front side (and on the back side, which is not shown in the figure). However, in terms of production, it is sometimes advantageous, and therefore preferred in variants of the present invention, if the structures are not made up to the edge, but the edges of the metal plates are continuous (this leads to less deformation during diffusion welding or the diffusion welding can be carried out with more pressure and/or less care at the edge).

Although it is preferred according to the invention to separate the individual segments 6 from one another, rearrange them and then use the new arrangement as a reactor, it is also possible to use the reactor 1a shown here as such in this form; in contrast to the more preferred embodiments according to the invention, such a reactor would have comparatively long or deep reactors (in the example shown, three times as long as when the segments are separated).

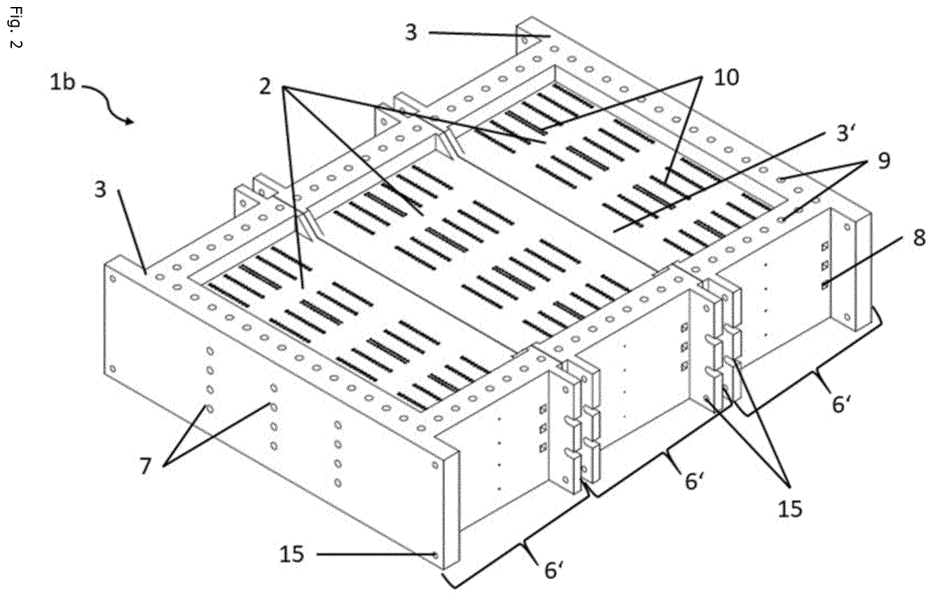

FIG. 2 shows a reactor 1b according to the invention, in which the individual segments 6 were separated from one another by being separated from one another at the blankings 4 (and the recesses 5 that are not shown) in the reactor 1a and then arranged again lying on top of one another. These segments, which are shown here lying horizontally, are now referred to as segments 6′ for the sake of distinction (but they are the same segments, only separated from each other). It can be clearly seen that the rearrangement of the segments results in many more reaction channels (reactor openings 10) being obtained from the reactor 1a. For reactors for fast reactions, the shallow depths with correspondingly short reaction channels are usually sufficient. The holes for heating elements 7 can also be seen here again. The inlet openings for the coolant 8 can also be seen again. In the reactors of the present invention, the channels for the reaction medium naturally run through the entire reactor 1a/1b and do not merely represent holes in the metal end metal plates 3. As 3′ the inner surfaces of the stacks 2 after milling down are shown in FIG. 2, where, as can be seen, the edges (in the picture, diagonally left at the top and diagonally right at the bottom) have been left standing so that a kind of surrounding edge is obtained, in which screw holes 9 have been machined (to which flanges, etc. can be attached; for example, a flange for a reaction fluid supply device). Due to the manufacturing by diffusion welding, the end metal plates 3 in this picture can no longer be clearly distinguished from the surrounding edge (transition not recognisable). Also clearly visible are various preparations for connections 15, which are designed as screw holes or notches here. These can be used to connect the individual segments 6′ to each other, or to connect the finished reactor 1b to other workpieces.

It should be noted that FIG. 2 shows a variant of the present invention in which the individual metal plates of the stacks 2 originally and during connection by diffusion welding had no structures at the edge; only after tilting (and preferably after joining) were the individual stack segments 6′ milled down to the depth of the reactor openings 10, leaving a circumferential edge (i.e. it was not milled on the outside), into which, for example, screw holes 9 (or similar) can then be worked (as shown here). In this way, the preceding diffusion welding can be carried out more easily/effectively and a uniform surface 3′ is obtained by the subsequent milling, so that the arrangement of the supply nozzles can then occur better and/or more precise and/or the distribution of the reaction medium can be more uniform; in this respect, this is a preferred variant of the present invention.

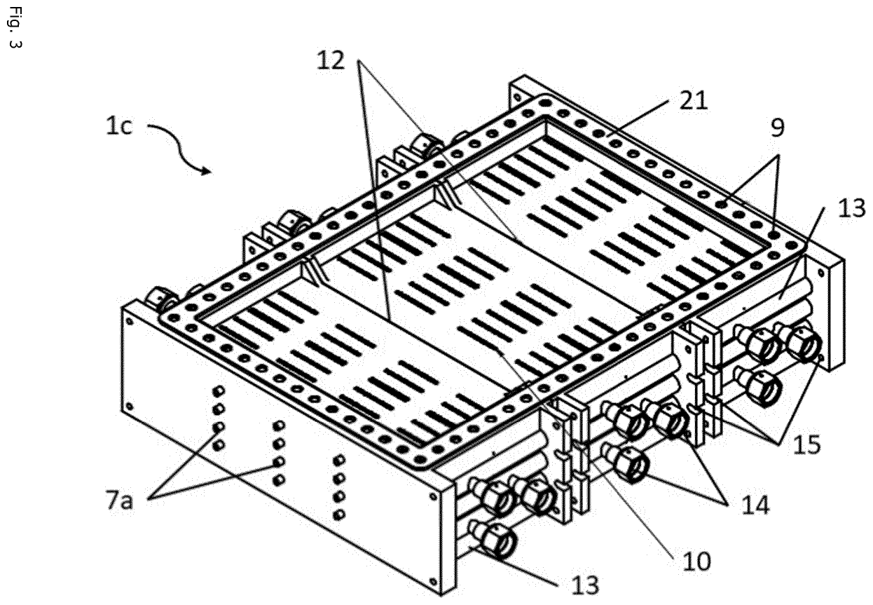

FIG. 3 shows a reactor 1c according to the invention, very similar to FIG. 2, but with slightly different details or representation. In contrast to FIG. 2, heating element ends can be seen here as 7a, of heating elements that have been pushed through the reactor (i.e. through the holes 7 for the heating elements). At the protruding ends of the heating elements 7a that can be seen in FIG. 3, depending on the design of the heating elements, for example, power connections can be provided for the possibility that the heating elements are based on electricity. In the embodiment according to FIG. 3, additionally a common flat sealing surface 21 is placed over the three individual segments, which is placed on the edges of the previously machined (milled down on the inside) segment surfaces 3′. In this common flat sealing surface 21, in turn, screw holes 9 can be seen, which of course match the screw holes 9 arranged below them, which have been worked into the partial/end metal plates or sides of the segments 6′. This sealing surface 21 can be made of the basically same material as the metal plates of the reactor or it can be made of other materials that are (particularly) suitable for sealing. In this respect, it is quite possible to use materials such as Teflon for this purpose. Furthermore, this figure illustrates a variant in which the inlet openings for coolant 8 (which can therefore no longer be seen in this figure) are connected to each other by (for example, welded-on) half-pipes 13. In this way, it is possible, for example, to supply both coolant inlet openings at the same height of a segment via just one coolant supply. For this purpose, for example, the (screw) connections for coolant supply 14 illustrated here can be used. Although FIG. 3 illustrates three half-pipes 13 arranged one above the other in each segment, with respective (screw) connections for coolant supply 14, these half-pipes can also be replaced by other configurations. For example, instead of the three individual half-pipes 13, it is just as possible to arrange a kind of hood over the side of each segment 6′, where this hood, for example, then covers all the inlet openings for the coolant 8 and this hood is then only provided with one (screw) connection for the coolant supply 14. Corresponding other configurations are also possible. In addition to the reactor openings 10 that can be seen again here in this figure, the connecting points between the individual segments 6′ can also be seen, here with the reference number 12. In this case, these represent circumferential welded connections with which the individual segments 6′ are connected. These circumferential welded connections 12 have, of course, been levelled by milling down the stack surfaces. In the event that the surfaces are not milled down, it is preferable to mill or level such circumferential welded connections in order to obtain a surface that is as even as possible (this is advantageous for further processing, handling safety or the distribution of reaction medium).

FIG. 4 shows an example of a structured metal plate 16 in a plan view. This is an exemplary, structured metal plate that can be used, for example, in the manufacture of the reactors 1a, 1b or 1c, with the difference that, for the sake of simplicity, only two segments 6 are shown in FIG. 4 and not three segments as in FIGS. 1, 2 and 3. The present invention is, of course, not limited to two segments or three segments 6, but the reduction to two or three segments 6 merely serves to simplify the illustration. Nevertheless, in various preferred variants of the present invention, it is preferred to use two segments 6′ or three segments 6. This figure shows various bores in the structured metal plate 16. These bores have two different diameters. The smaller diameters, twelve of which can be seen per segment (in three groups of four) are illustrated with the number 7 and correspondingly represent the holes for heating elements 7. These holes therefore pass through all the structured metal plates to be arranged one above the other at the same location; the corresponding structured metal plates 16 to be employed (and others) are to be manufactured accordingly and it is to be ensured that the bores are superimposed—if heating elements are to be passed through the reactor. In this context, it should be noted that, in the context of the present invention, it is not mandatory for heating elements to be passed through the reactor; this is merely one (of many possible) variants, insofar the bores/holes 7 are optional in the context of the present invention and are only absolutely necessary if heating elements are to be used. Regardless of whether heating elements are actually used, however, it is preferable in some variants to provide the holes 7 in order to be flexible in the use of heating elements (in addition, in some variants, (heated) air can simply be passed through the holes or the channels formed from them). The other, larger bores with the reference number 11a represent openings for coolant channels perpendicular to the plane layer; the channels themselves are formed when several structured plates, which are not necessarily identical, are placed on top of each other—also in this regard, it has to be ensured that the plates placed on top of each other are matched or adapted to each other with regard to the structures for the coolant (channels) (this is clear to those skilled in the art). Coolant can then flow through these openings 11a (if used—applications are also conceivable in which no coolant has to be passed through—the reactors according to the invention can of course also be used for such applications). Also shown in this figure are the blankings 4 and the recesses 5 along which the two segments 6 then are (can be) separated from each other. The recesses 5 have a corresponding recess length A, which in the example shown (a preferred embodiment) essentially corresponds to the width B of a single reactor module, i.e. the respective reactor channels arranged next to each other of a section. In this figure, designated with R, also the reaction zone length of the respective reactor modules is shown. Furthermore, T indicates the depth of the respective segments 6. The figure shows a structured metal plate 16 in which the structuring does not extend to the edge (upper and lower edge in the figure) (the depth T of the segments 6 is significantly greater than the reaction zone length R of the individual reactor modules), so that after the separation of the individual segments 6 from one another (the tilting by 90°) and the joining of the segments, the surface of the stacks must, in this case, then be milled down until the reactor openings are reached. This is a preferred embodiment of the present invention, but (as already mentioned) it is quite possible within the scope of the present invention to extend the structures of the individual reactor modules in the structured metal plates to the respective edge of the metal plates, in which case milling down would no longer be necessary.

It should be noted that, in the context of the present invention, it is not mandatory that the surfaces of the segments 6′ be machined by milling to reach the openings of the reactor modules. Other known methods of surface removal can be used as well. However, milling is usually preferred for practical reasons.

FIG. 5 shows, in an oblique view from above, various structured metal plates (16, 17, 18, 19, 20) how they can be arranged on top of each other to form (part of) a stack 2 made of structured metal plates. The structured metal plate 16 corresponds to the one shown in FIG. 4. In the context of FIG. 5, it is also illustrated for this structured metal plate 16 with L′ where the reaction fluid will flow along (in the finished reactor, from bottom to top along the linear reactor module structure). Here, too, in the various structured metal plates, the recesses 5 and blankings 4 are illustrated along which the individual segments 6 are later separated from one another. The structured metal plate 20 represents a transition plate that can be structured differently as required, so that in this example, a precise structuring is not shown. As 11a bores or coolant channels perpendicular to the respective plate layer are again shown. Additionally, as 11b coolant channels in the respective plate layer are shown. In the structured metal plate 19, the coolant channels 11a are, perpendicular to the plate layer, connected to the coolant channels 11b lying in the plate layer. In this way, coolant can enter the layer through coolant channel 11a and then flow along the coolant channels 11b in the layer. Via this coolant channel 11b, the coolant can then get into the next layer, in this case the structured metal plate 18 (in the illustrated variant, corresponding structures lying in the plate layer are also provided in the metal plate 18-but this is not necessarily always the case). In the then following structured metal plate 17 an area of the longitudinal flow of the coolant is illustrated with L, i.e. in this plate the coolant is deflected and then flows, for example, vertically in the plate layer. In this respect, it is possible, with appropriately designed structured metal plates, to introduce the coolant into the stack 2 of structured metal plates coming from a bottom side (or top side) of the reactor, to then deflect it in different directions by means of various deflections in the respective plate layers, in order to achieve a coolant distribution as good as possible. In doing so, cross-flows (partly) can be provided, which are illustrated here with K for a cross-flow area. It should be noted that the illustrated sequence of structured metal plates or the exact structure of the metal plates shown here are only exemplary and that the present invention is, of course, not limited to the sequences of structured metal plates and the illustrated structures illustrated here.

### LIST OF REFERENCE SIGNS

1a reactor (before separation of the segments)
1b reactor (after separation and rearrangement of the segments)
1c reactor (after separation and rearrangement of the segments; other design)
2 stack of structured metal plates
3 metal end metal plates (non-structured, only provided with inlet openings for coolant)
3′ stack surfaces after milling down
4 blankings
5 recesses
6 segments
6′ segments, horizontal (after separation)
7 holes for heating elements
7a (visible parts of the) heating elements
8 inlet openings for coolant
9 screw holes (for the attachment of flanges)
10 reactor openings
11a coolant channel perpendicular to the plate layer
11b coolant channel in the plate layer
12 circumferential welded connection
13 half-pipes (welded on, connecting the inlet openings for coolant)
14 (screw) connections for coolant supply
15 provisions for connections (screw holes; notches etc.)
16 structured metal plate
17 structured metal plate
18 structured metal plate
19 structured metal plate
20 structured metal plate
21 common planar sealing surface (with screw holes 9)
A recess length
B width of reactor module
K cross-flow section
L longitudinal flow of the coolant
L′ longitudinal flow of the reaction fluid
R reaction zone length
T depth of the segments 6

### Example

The invention will now be further explained with reference to the following non-limiting example.

A reactor according to the invention, in which the welded reactor plate stack before separation of the segments had a segment width of 530 mm, a depth of 580 mm for a total of three segments, and a stack height with end plates of 277 mm, was produced. In this case, a thickness of the end plates (3) of 50 mm was employed. Into each segment four reactor modules are integrated, which have a heat transfer area of 70 mm wide and 120 mm long in the milled end state. The length of the four recesses (5) per segment was 72 mm each, resulting in a ratio of recesses to segment width of about 60%, taking into account the blankings (4) at the edge of the plates. The width of the recesses was 5 mm. The plate arrangement was regular, so that upper and lower end plate were selectively combinable for the welding by electron beam welding. Into each of the two end plates three inlet openings (8), each with a diameter of 11.3 mm, per segment were incorporated, which were 5.3 mm away from the plate stack. From there, the cooling water flow was distributed to coolant channels perpendicular to the plate layer (11a) with a diameter of 9 mm. The coolant channels in the plate layer (11b) were 3 mm wide and 3 mm deep. The cooling channels running parallel to the reaction channels were 1.5 mm wide and 0.5 mm deep. The reaction channels were 4×4 mm. The prefabricated holes for heating elements (heating cartridges) (7) were 6 mm in diameter and were drilled out to 10 mm in diameter. After diffusion welding, the plate stack was only about 270 mm high. Thus, after arranging the segments in the rotated state, the total height (three times segment 6′) of the finished reactor was 810 mm, with a known width of the segments of 530 mm. In the assembly, after rearrangement of the segments, the distance between the front side and back side of the segments was about 193 mm (depth of segments 6). Thus, the ratio of the stack height ultimately present in the finished reactor to the segment depth was about 5.2. The reactor was finally milled on both sides over its entire surface, so that the maximum depth of the finished reactor was 170 mm. To open the reaction channels, an additional 25 mm was milled on both sides, resulting in a total length of the reaction channels of 120 mm. The circumferential edge was used for stud bolts that fix a continuous flange. The reactor was filled with nickel catalyst in a grain size of 300-400 um diameter and used for methanisation under cooling with fully demineralised water.

1-13. (canceled)

14. A method of producing a reactor which comprises planes for a flow of reaction medium and a flow of heat transfer medium, wherein the method comprises
(a) stacking structured metal plates, each of same width and same depth, on top of each other for respective planes;
(a1) optionally, stacking one or two structured or non-structured end metal plates as uppermost and/or lowermost end plates;
(b) joining the stacked plates to each other by diffusion welding;
(c) separating the diffusion-welded stack thus obtained in a width direction into individual segments of equal depth;
(d) placing a desired number of individual segments obtained in (c) on top of and/or next to one another and joining the segments together.

15. The method of claim 14, wherein prior to or simultaneously with (c) recesses are incorporated into the metal plates or the stack, along which recesses a separation then takes place in (c).

16. The method of claim 15, wherein a ratio of the sum of recess lengths to a total width of the metal plates is from 60% to 90%.

17. The method of claim 15, wherein a ratio of segment depth to recess length is from 2 to 8.

18. The method of claim 14, wherein the metal plates used in (a) are approximately square.

19. The method of claim 14, wherein connection points of individual segments obtained in (d) are planarized to produce planar sealing edges.

20. The method of claim 19, wherein, in the case of circumferential welding in (d), the individual segments are planarized by milling resulting weld seams.

21. The method of claim 19, wherein planar sealing edges are adapted by means of a flange.

22. The method of claim 14, wherein the metal plates are selected and structured such that a coolant in the resulting reactor flows at least partially in cross-current transverse to a flow direction of a process fluid.

23. The method of claim 22, wherein at least two deflections of the coolant occur within distribution channels.

24. The method of claim 14, wherein the metal plates are selected and structured such that a coolant in the resulting reactor flows in cross-current through areas without proximity to reaction channels.

25. The method of claim 14, wherein structured or non-structured end metal plates, if present, have a thickness of at least 10 mm and comprise inlet openings for coolant.

26. The method of claim 25, wherein the inlet openings are on sides of the plates.

27. The method of claim 14, wherein the method further comprises after (c) or after (d),
(f) milling down front and back sides of the segments to openings of reaction channels.

28. The method of claim 27, wherein (f) is carried out after (d).

29. A reactor manufactured according to the method of claim 14.

30. The reactor of claim 29, wherein the reactor comprises a desired number of individual segments of diffusion-welded stacks of micro-structured metal plates, arranged on top of and/or next to one another and connected to one another, and distribution channels for the coolant are deflected at least twice.

31. The reactor of claim 30, wherein
the coolant is initially introduced laterally in cross-flow exclusively into areas resulting from end metal plates of the stacks, transversely to a flow direction of the process fluid, and
thereafter the coolant is deflected in stack direction and distributed over several reaction areas within a segment.

32. A process for carrying out a Fischer-Tropsch reaction, wherein the method comprises using the reactor of claim 29.

33. A process for carrying out a methanisation reaction, wherein the method comprises using the reactor of claim 29.
